(19) United States
(12) Patent Application Publication
(10) Pub. No.: US 2023/0293857 A1
(43) Pub. Date: Sep. 21, 2023

(54) Handheld, Untethered Catheter Containment System and Method

(71) Applicant: Bard Access Systems, Inc., Salt Lake City, UT (US)

(72) Inventors: Luke Robbins, Egham (GB); Christopher Vincent, London (GB); Norihiko Inoue, London (GB); Kyle G. Thornley, Farmington, UT (US); Glade H. Howell, Draper, UT (US); Joe Spataro, Cottonwood Heights, UT (US); Eric W. Lindekugel, Salt Lake City, UT (US); Lisa I. Bailey, Millcreek, UT (US); Jonathan W. Rutledge, Layton, UT (US); David Brashares, West Jordan, UT (US); Hannah Gracie O'neill, Millcreek, UT (US)

(21) Appl. No.: 18/123,778

(22) Filed: Mar. 20, 2023

Related U.S. Application Data
(60) Provisional application No. 63/322,122, filed on Mar. 21, 2022.

(51) Int. Cl. A61M 25/09; A61M 25/00

(52) U.S. Cl. CPC A61M 25/09; A61M 25/0097; A61M 2025/09116

(57) ABSTRACT

A catheter containment device configured to retain a proximal portion of a catheter placement system, such as a catheter, hub, extension leg, luer lock, or guidewire. The device includes a handle configured to engage a digit of the clinician to couple the device and the proximal portion of a catheter placement system thereto. This allows the clinician to retain the proximal portion within the sterile field without reducing dexterity or obstructing the clinician's view of the insertion site or the catheter placement system. Further the device can allow the clinician to manipulate the proximal portion of the catheter system while mitigating dropping or tangling.

### PRIORITY

This application claims the benefit of priority to U.S. Provisional Application No. 63/322,122, filed Mar. 21, 2022, which is incorporated by reference in its entirety into this application.

### BACKGROUND

Central venous catheters (“CVCs”) are commonly introduced into patients and advanced through their vasculatures by way of the Seldinger technique. The Seldinger technique utilizes a number of steps and medical devices (e.g., a needle, a scalpel, a guidewire, an introducer sheath, a dilator, a CVC, etc.). While the Seldinger technique is effective, the number of steps are time consuming, handling the number of medical devices is awkward, and both of the foregoing can lead to patient trauma and/or increased risk of infection. There is a relatively high potential for touch contamination due to the number of medical devices that need to be interchanged during the Seldinger technique. As such, advanced catheter placement systems have been developed to reduce the number of steps and medical devices involved in placing a catheter, such as a CVC, into a patient.

Exemplary advanced catheter placement systems can include, for example, rapidly insertable central catheter (RICC) placement systems. The RICC includes a mono-luminal access section, disposed distally and having a first diameter, a multi-luminal catheter section disposed proximally and having a second diameter larger than then first diameter, and a tapered dilation section disposed therebetween and extending between the first diameter and the second diameter.

Whether using a traditional Seldinger techniques or using RICC placement systems, often the clinician is required to stabilize a proximal portion of the catheter placement system, (e.g., hub, extension legs, guidewires, etc.) while placing a distal tip of the catheter within the vasculature. This prevents these proximal portions of the catheter placement system from tangling or falling outside of the sterile field. However, holding these proximal portions can reduce the dexterity of the clinician and/or become entangled, complicating the procedure. Further these proximal portions are at risk from falling outside of the sterile field, contaminating the device and causing the clinician to abort the placement procedure. What is needed therefore is a catheter containment system that allows a clinician to secure the proximal portions of the catheter placement system during placement, without reducing the dexterity of the clinician and mitigating these proximal portions from falling outside of the sterile field. Embodiments disclosed herein are directed to address at least the foregoing.

### SUMMARY

Disclosed herein is a catheter containment device for retaining a portion of a catheter placement system including, a body defining a channel extending longitudinally along a first axis, the channel configured to retain the portion of the catheter placement system, and having an opening extending longitudinally and configured to allow ingress or egress along a second axis extending at an angle to the first axis of the channel, and a handle coupled to the body and configured to be grasped by a digit of a clinician.

In some embodiments, the portion of the catheter placement system includes one of a catheter body, catheter hub, extension leg, luer lock, or guidewire.

In some embodiments, the body includes a first channel configured to retain a first extension leg and a second channel configured to retain a second extension leg.

In some embodiments, the body includes a first channel configured to retain a first extension leg and a second extension leg.

In some embodiments, the body includes a first channel extending along the first axis of the channel, and a second channel extending along a third axis at an angle to the first axis of the channel.

In some embodiments, an inner diameter of the channel is equal to or slightly smaller than an outer diameter of the portion of the catheter placement system.

In some embodiments, a width of the opening of the channel is smaller than an outer diameter of the portion of the catheter placement system.

In some embodiments, one or both of the body and the handle is formed of a substantially rigid, or resilient material selected from a group consisting of a plastic, polymer, metal, alloy, or composite.

In some embodiments, one or both of the body and the handle is formed of a flexible, malleable, or elastically deformable material selected from a group consisting of a plastic, polymer, elastomer, rubber, silicone rubber, metal, alloy, shape memory material, super-elastic material, Nitinol, composite.

In some embodiments, the handle is formed of a first material having a substantially rigid, or resilient, material properties and including a second material disposed thereon having a relatively softer, or more flexible material properties.

In some embodiments, the handle is a ring defining an aperture and configured to receive one or more digits therethrough.

In some embodiments, the handle is hingedly, rotatably or pivotally coupled to the body.

In some embodiments, the handle extends from the body at an angle relative to the first axis of the channel.

In some embodiments, the catheter containment device further includes a first handle extending from the body in a first direction and a second handle extending from the body in a second direction, opposite the first direction.

In some embodiments, the handle includes one of a plug or a cross bar disposed at an opposite end of the handle from the body.

Also disclosed is a catheter containment device for retaining a portion of a medical device including, a body defining a disc shape extending over a horizontal plane and defining a top surface and a bottom surface, and a device channel extending along an axis, parallel to one of the top surface or the bottom surface, and including an opening communicating with one of the top surface or the bottom surface, the device channel configured to receive a portion of the medical device therein.

In some embodiments, the body further includes a bi-concave cross-section having one of a first finger channel extending over the top surface and a second finger channel extending over the bottom surface, one of the first finger channel or the second finger channel extending at an angle relative to the device channel.

In some embodiments, the device channel is configured to retain one of a catheter body, catheter hub, extension leg, luer lock, or guidewire.

In some embodiments, the body includes a first device channel configured to retain a first extension leg and a second device channel extending parallel to the first device channel and configured to retain a second extension leg.

In some embodiments, the first device channel is configured to retain a first extension leg and a second extension leg.

In some embodiments, an inner diameter of the device channel is equal to or slightly smaller than an outer diameter of the portion of the medical device.

In some embodiments, a width of the opening of the device channel is smaller than an outer diameter of the portion of the medical device.

In some embodiments, the body is formed of a substantially rigid, or resilient material selected from a group consisting of a plastic, polymer, metal, alloy, or composite.

In some embodiments, the body is formed of a flexible, malleable, or elastically deformable material selected from a group consisting of a plastic, polymer, elastomer, rubber, silicone rubber, metal, alloy, shape memory material, super-elastic material, Nitinol, composite.

In some embodiments, the body is formed of a first material having a substantially rigid, or resilient, material properties and including a second material disposed thereon having a relatively softer, or more flexible material properties.

Also disclosed is a method of placing a catheter including, providing a catheter placement system including, a needle, a blood flash system, a catheter including, a catheter body, a hub disposed at a proximal end of the catheter body, and an extension leg extending proximally from the hub, and a guidewire extending through a lumen of the catheter, securing a portion of the catheter or the guidewire in a channel of a containment device, engaging the catheter containment device with a digit of a first hand to retain the catheter containment device and the portion of the catheter or the guidewire secured therewith, and manipulating the catheter placement system with the first hand to place the catheter within a vasculature.

In some embodiments, the portion of the catheter includes a proximal portion of the catheter body, the catheter hub, the extension leg, a luer lock, or a proximal portion of the guidewire.

In some embodiments, the catheter containment device includes a body defining the channel and a handle configured to engage the digit of the first hand.

In some embodiments, the method further includes engaging the portion of the catheter or the guidewire with the channel in one of an interference fit, press-fit, or snap-fit engagement.

In some embodiments, the handle is formed of a first material having a substantially rigid, or resilient, material properties and including a second material disposed thereon having a relatively softer, or more flexible material properties.

In some embodiments, the handle is a ring defining an aperture and configured to receive one or more digits therethrough.

In some embodiments, the step of engaging further includes compressing a first handle with a first digit and a second handle with a fourth digit of the first hand.

In some embodiments, the step of engaging further includes applying a radially outward pressure against a first cross bar of a first handle with a first digit of the first hand, and against a second cross bar of a second handle with a fourth digit of the first hand.

In some embodiments, the step of engaging further includes compressing one of the body, the handle or a cross bar coupled to the handle, between two or more digits of the first hand.

Also disclosed is a catheter containment device for retaining a portion of a catheter placement system including, a body defining a channel extending along a longitudinal axis, the channel configured to permanently retain the portion of the catheter placement system, and a handle coupled to the body and configured to be grasped by a digit of a clinician.

In some embodiments, the portion of the catheter placement system includes a proximal end of a guidewire permanently affixed to the body.

In some embodiments, the portion of the catheter placement system includes a proximal portion of a guidewire slidably engaged with the body.

### DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1A shows a perspective view of a catheter placement system in an unfolded configuration, in accordance with embodiments disclosed herein.

FIG. 1B shows a plan view of a catheter placement system in a folded configuration ready for use, in accordance with embodiments disclosed herein.

FIG. 2 shows a side view of a catheter of the catheter placement system of FIG. 1A in an unfolded configuration, in accordance with embodiments disclosed herein.

FIG. 3A shows close up detail of a distal portion of the catheter of FIG. 2, in accordance with embodiments disclosed herein.

FIGS. 3B-3C show cross-section views of the catheter of FIG. 3A, in accordance with embodiments disclosed herein.

FIGS. 4A-4B show a catheter containment system in an exemplary environment of use, in accordance with embodiments disclosed herein.

FIG. 4C shows a side view of the catheter containment system of FIG. 4A, in accordance with embodiments disclosed herein.

FIG. 5A shows a side view of a catheter containment system, in accordance with embodiments disclosed herein.

FIG. 5B shows a catheter containment system in an exemplary environment of use, in accordance with embodiments disclosed herein.

FIG. 5C shows a perspective view of a catheter containment system, in accordance with embodiments disclosed herein.

FIGS. 5D-5F show a catheter containment system in exemplary environments of use, in accordance with embodiments disclosed herein.

FIG. 6A shows a perspective view of a catheter containment system, in accordance with embodiments disclosed herein.

FIG. 6B shows a perspective view of a catheter containment system, in accordance with embodiments disclosed herein.

FIG. 7A shows a perspective view of a catheter containment system, in accordance with embodiments disclosed herein.

FIG. 7B shows a side view of the catheter containment system of FIG. 7A, in accordance with embodiments disclosed herein.

FIG. 7C shows the catheter containment system of FIG. 7A in an exemplary environment of use, in accordance with embodiments disclosed herein.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

In the following description, the terms “or” and “and/or” as used herein are to be interpreted as inclusive or meaning any one or any combination. As an example, “A, B or C” or “A, B and/or C” mean “any of the following, A, B, C, A and B, A and C, B and C, A, B and C.” An exception to this definition will occur only when a combination of elements, components, functions, steps or acts are in some way inherently mutually exclusive.

With respect to “proximal,” a “proximal portion” or a “proximal end portion” of, for example, a catheter disclosed herein includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal end portion, or the proximal length of the catheter can include the proximal end of the catheter; however, the proximal portion, the proximal end portion, or the proximal length of the catheter need not include the proximal end of the catheter. That is, unless context suggests otherwise, the proximal portion, the proximal end portion, or the proximal length of the catheter is not a terminal portion or terminal length of the catheter.

With respect to “distal,” a “distal portion” or a “distal end portion” of, for example, a catheter disclosed herein includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal end portion, or the distal length of the catheter can include the distal end of the catheter; however, the distal portion, the distal end portion, or the distal length of the catheter need not include the distal end of the catheter. That is, unless context suggests otherwise, the distal portion, the distal end portion, or the distal length of the catheter is not a terminal portion or terminal length of the catheter.

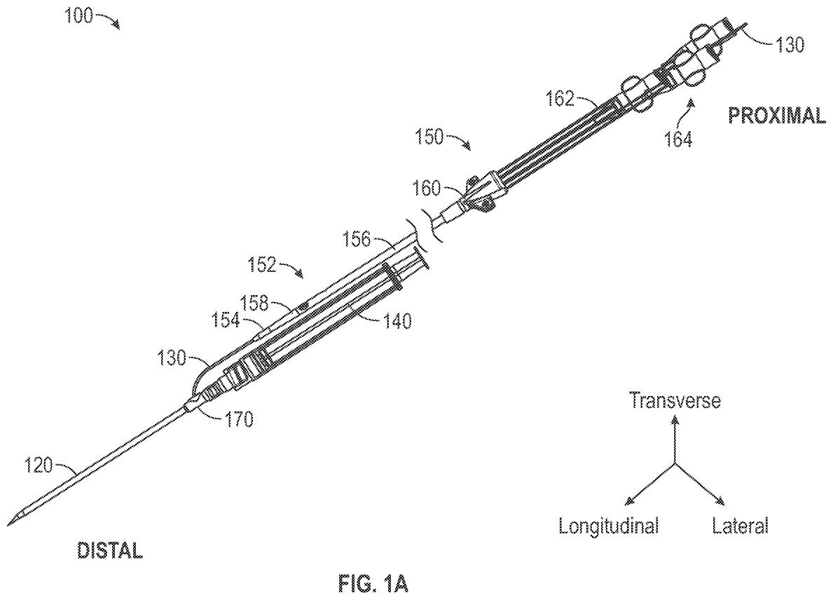

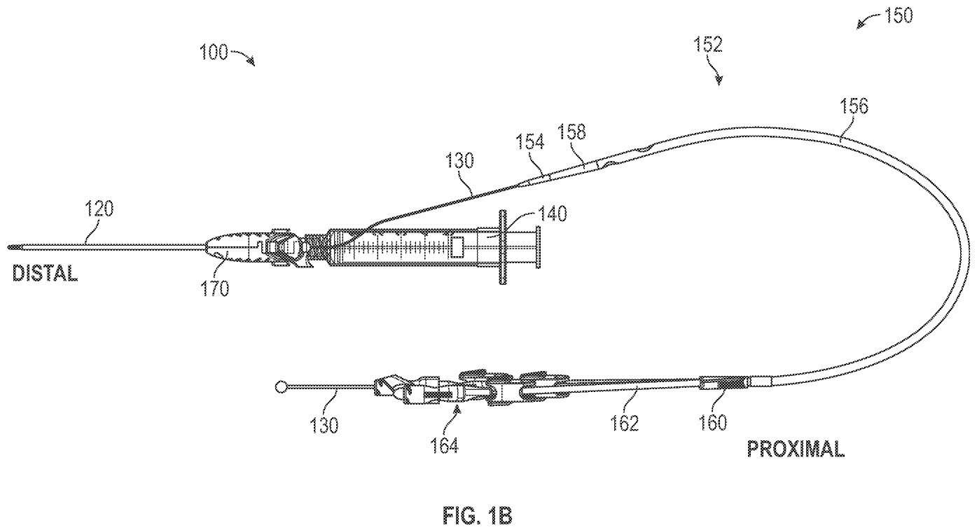

To assist in the description of embodiments described herein, as shown in FIG. 1A, a longitudinal axis extends substantially parallel to an axial length of the catheter. A lateral axis extends normal to the longitudinal axis, and a transverse axis extends normal to both the longitudinal and lateral axes. A horizontal plane can be defined by the lateral axis and the longitudinal axis. A vertical plane extends normal to the horizontal plane.

Unless defined otherwise, all technical and scientific terms used herein have the same meaning as commonly understood by those of ordinary skill in the art.

FIGS. 1A-1B show an exemplary catheter placement system (“system”) 100, and generally including a needle 120, a guidewire 130, a blood flash system 140, a catheter 150, and a needle housing (“housing”) 170. FIG. 1A shows the system 100 in an unfolded configuration for ease of illustration. FIG. 1B shows a plan view of the system 100 in a folded configuration ready for use. In an embodiment the catheter placement system 100 can be a Rapidly Insertable Central Catheter (RICC) placement system 100 configured to place a RICC 150. However, it will be appreciated that other catheter placement systems configured to place other types of catheters are also contemplated. Exemplary catheters 150 can also include peripheral intravenous (PIV) catheters, peripherally inserted central catheter (PICC), central venous catheters (CVC), midline catheters, dialysis catheters, single lumen catheters, multi-lumen catheters, or the like.

In an embodiment, the catheter 150 generally includes a catheter body 152 supported at a proximal end by a catheter hub (“hub”) 160. The hub 160 includes one or more extension legs 162 extending proximally therefrom and can include a fluid coupling device, such as a luer lock 164, disposed at a proximal end of one of the one or more extension legs 162. The luer lock 164 is configured to couple the extension leg 162 with a medical fluid line, syringe, or the like. Each extension leg of the one or more extension legs 162 is in fluid communication with a lumen of the catheter body 152. For example, a first extension leg 162A is in fluid communication with a proximal lumen 114A, a second extension leg 162B is in fluid communication with a medial lumen 114B, and a third extension leg 162C is in fluid communication with a distal lumen 114C. The catheter body 152 includes an access section 154 disposed distally, a catheter section 156 disposed proximally, and a dilation section 158 disposed therebetween. The access section 154 defines a single lumen and has a first outer diameter, the catheter section 156 defines two or more lumen and has a second diameter larger than the first diameter. The dilation section 158 disposed between the access section 154 and the catheter section 156 defines a tapered outer profile extending from the first diameter of the access section 154 to the second diameter of the catheter section 156. A guidewire 130 can extend through a lumen of the catheter 150 from a proximal end of an extension leg 162, to a distal tip of the access section 154.

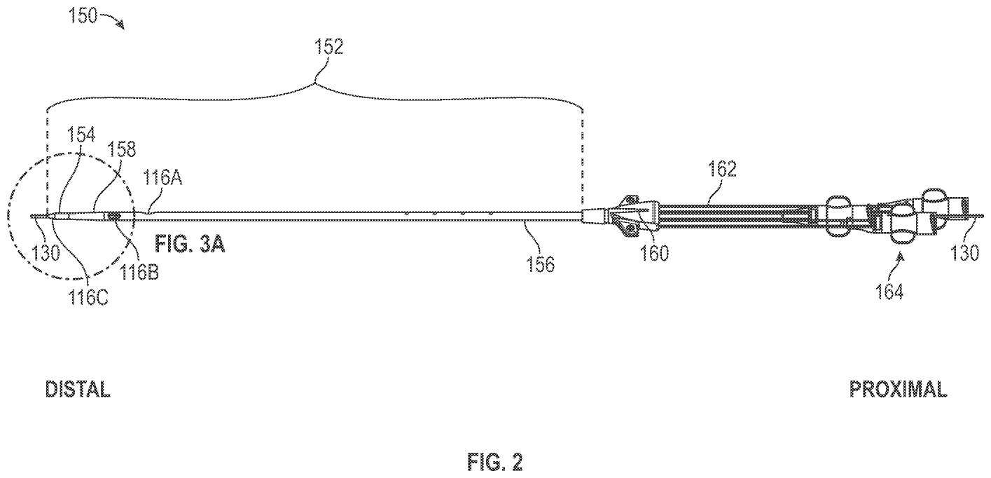

FIG. 2 shows further details of an exemplary catheter 150 of the system 100. In an embodiment, different sections of the catheter 150 are required to perform different functions and as such are required to display different mechanical properties. For example, the access section 154 and the dilation section 158 can provide a more rigid mechanical properties or harder durometer material relative to the catheter section 156. As such, the access section 154 and dilation section 158 can withstand greater axial forces without kinking or collapsing, as these sections are urged distally, forming and dilating the insertion site. The catheter section 156 can be formed of a softer durometer, or a more compliant material to facilitate negotiating the catheter section 156 through tortuous vascular pathways.

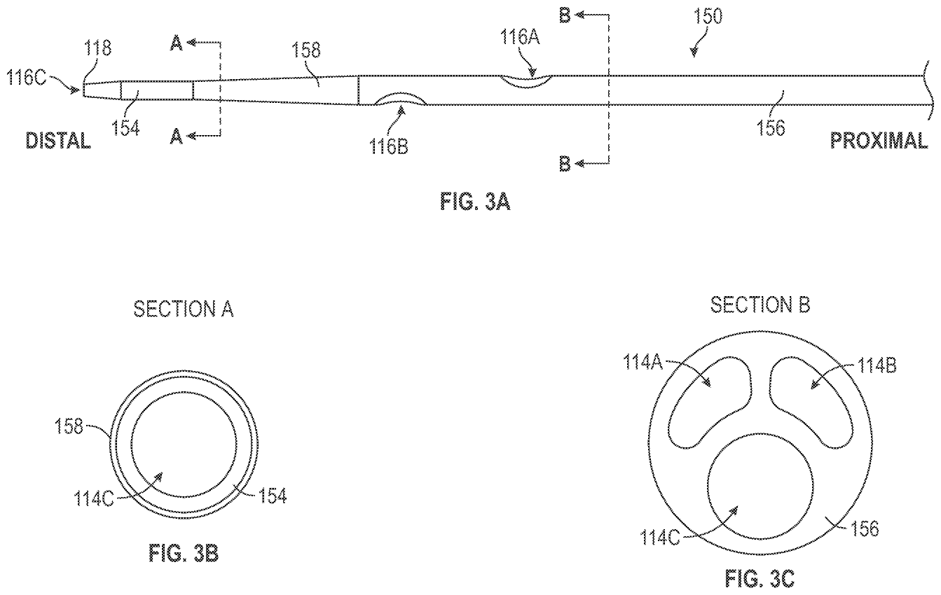

FIGS. 3A-3C show further details of a distal portion of the catheter 150, including the access section 154, the catheter section 156, and the dilation section 158. In an embodiment, the catheter section 156 includes a proximal lumen 114A terminating at a proximal lumen aperture 116A, and a medial lumen 114B terminating at a medial lumen aperture 116B. In an embodiment, each of the proximal lumen aperture 116A and the medial lumen aperture 116B extends through a side wall of the catheter section 156. In an embodiment, each of the proximal lumen aperture 116A and the medial lumen aperture 116B are disposed proximally of the dilation section 158. In an embodiment, the proximal lumen aperture 116A can be disposed proximally of the medial lumen aperture 116B. In an embodiment, the proximal lumen aperture 116A and the medial lumen aperture 116B can be disposed equidistant from a distal tip of the catheter 150.

FIG. 3B shows a cross section view of the catheter body 152 at point “A” of FIG. 3A. As shown, the access section 154 defines a single lumen and a relatively smaller outer diameter. In an embodiment, a proximal portion of the access section 154 is received within a distal portion of the dilation section 158. A distal lumen 114C of the catheter 150 can extend to a distal tip 118 of the catheter 150 and communicates with a distal lumen aperture 116C. FIG. 3C shows a cross section view of the catheter section 156 at point “B” of FIG. 3A, showing the proximal lumen 114A, medial lumen 114B and distal lumen 114C.

Further details and embodiments of such catheter placement systems 100 can be found, for example, in U.S. Pat. Nos. 11,517,719, 10,376,675, U.S. 2019/0255294, U.S. 2021/0069471, U.S. 2021/0113809, U.S. 2021/0113810, U.S. 2021/0121667, U. S. 2021/0121661, U. S. 2021/0228843, U. S. 2021/0283381, U. S. 2021/0322729, U. S. 2021/0361915, U. S. 2021/0330941, U. S. 2021/0330942, U. S. 2021/0402149, U. S. 2022/0001138, U. S. 2021/0402142, U. S. 2022/0032013, U. S. 2021/0402153, U. S. 2021/0379336, U. S. 2021/0283368, U. S. 2022/0062528, U. S. 2022/0032014, U. S. 2022/0126064, U. S. 2022/0193378, U. S. 2022/0176081, U. S. 2022/0193376, U. S. 2022/0193377, U. S. 2022/0152368, U. S. 2022/0176082, U. S. 2022/0193379, U.S. 2022/0296862, U.S. 2022/0323723, U.S. 2022/0370762, U.S. 2022/0362524, U.S. 2023/0043989, U.S. 2023/0041261, U.S. 2023/0039733, U.S. 2023/0042898, each of which is incorporated by reference in its entirety into this application.

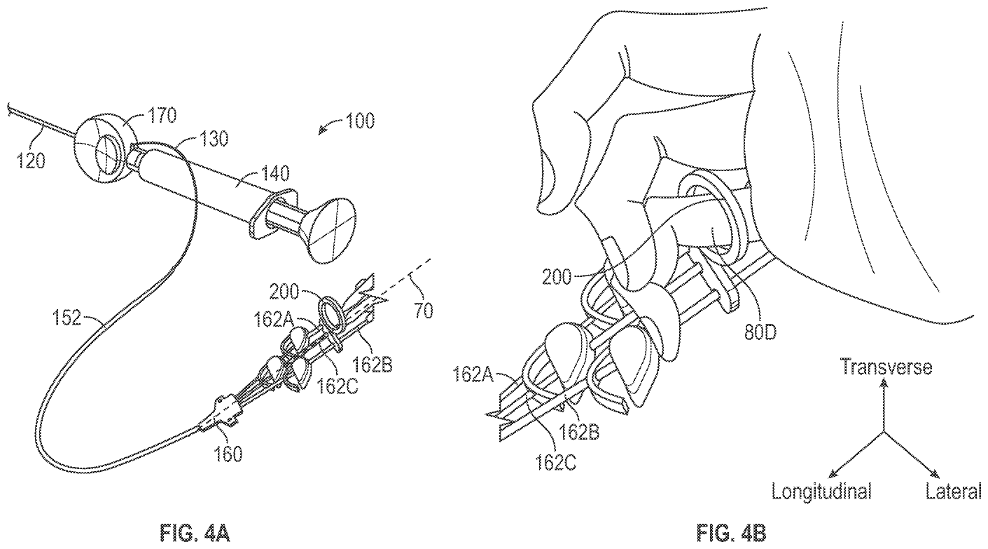

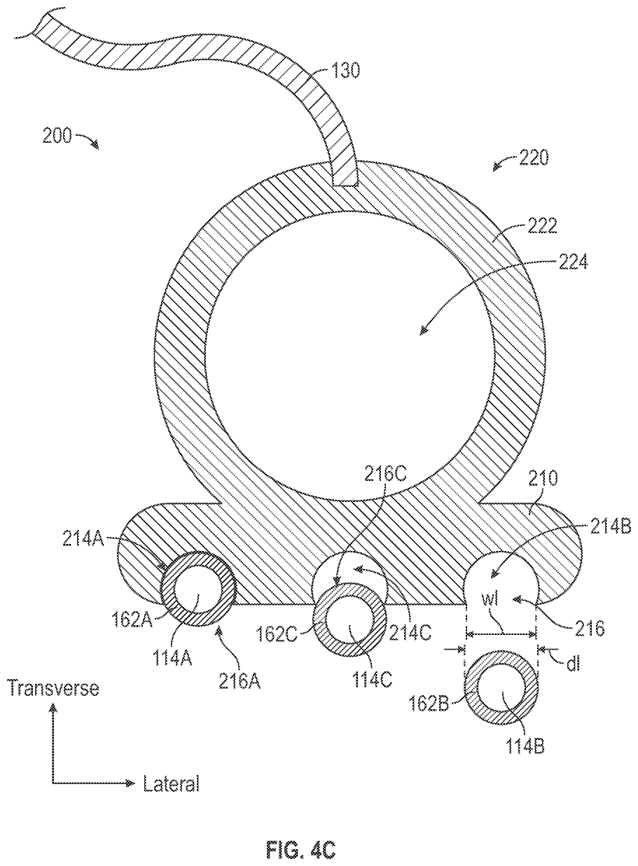

FIGS. 4A-4B show a catheter containment device (“device”) 200 in an exemplary environment of use, coupled to an extension leg 162 of a catheter placement system 100. FIG. 4C shows a side view of the catheter containment device 200 of FIG. 4A. The device 200 generally includes a body 210 configured to retain a portion of the catheter placement system 100, for example a portion of the catheter body 152, hub 160, one or more extension legs 162, luer lock 164, guidewire 130, or combinations thereof. The device 200 further includes a handle 220 configured to be grasped by the clinician. In an embodiment, a portion of the catheter placement system 100 is permanently affixed to the device 200, e.g., one of the body 210 or the handle 220. For example, a proximal end of the guidewire 130 can be formed integrally with, or permanently coupled to, the handle 220 and/or the body 210. Advantageously, this can prevent the guidewire 130 from also be drawn through the catheter placement system 100 and into the vasculature, as well as preventing the guidewire 130 from falling outside of the sterile field.

In an embodiment, as shown in FIG. 4C, the handle 220 can comprise a ring 222, defining an aperture 224 and configured to receive a digit 80 of a clinician therethrough. In an embodiment, the ring 222 can define a circular, oval, ellipsoidal, or polygonal shaped aperture 224. However, it will be appreciated that other regular or irregular, closed curve shapes are also contemplated. In an embodiment, the aperture 224 can be configured to receive two or more digits 80 therethrough. In an embodiment, the handle 220 can comprise two or more rings 222, each configured to receive one or more digits 80 therethrough. In an embodiment, the ring 222 is formed of a substantially rigid, or resilient material such as a plastic, polymer, metal, alloy, composite, or the like. In an embodiment, the ring 222 can be formed of a flexible, malleable, or elastically deformable material such as a plastic, polymer, elastomer, rubber, silicone rubber, metal, alloy, shape memory material, super-elastic material, Nitinol, composite, or the like.

Advantageously, the clinician can elastically deform, or plastically deform, the handle 220 to fit over one or more digits 80 and retain the device 200 thereon in a preferred position and remain in position until repositioned. For example, as shown in FIG. 4B, the device 200 can engage a little finger digit 80D and can be rotated such that the body 210 of the device 200 is positioned outwards from the clinician's palm. The clinician can elastically or plastically deform the handle 220 to retain the device 200 in this position relative to the hand. In an embodiment, one or both of the handle 220 and the body 210 can be formed of an elastic material, super-elastic material, or shape memory material, such as Nitinol, or the like, and can be configured to resume the original shape of the handle 220 once the clinician has finished with the procedure. In an embodiment, the handle 220 and the body 210 can be hingedly or rotatably coupled to allow the handle 220 to rotate or pivot relative to the body 210.

With continued reference to FIG. 4C, in an embodiment, the body 210 includes one or more channels 214 formed therein and extending along a longitudinal axis 70. The channel 214 is configured to releasably retain a portion of the catheter placement system 100, such as a portion of the catheter body 152, hub 160, extension leg 162, luer lock 164, guidewire 130, combinations thereof, or the like. For example, as shown in FIGS. 4A-4C, the body 210 includes a first channel 214A configured to receive a first extension leg 162A, a second channel 214B configured to receive a second extension leg 162B, and a third channel 214C configured to receive a third extension leg 162C. However, this is not intended to be limiting and the body 210 can include one or more channels 214 configured to retain other portions of the catheter placement system 100. In an embodiment, one or both of the body 210 and the handle 220 are formed of a substantially rigid, or resilient material such as a plastic, polymer, metal, alloy, composite, or the like.

In an embodiment, a portion of the catheter placement system 100, e.g., extension leg 162, guidewire 130, etc. can be permanently retained within a channel 214 of the body 210. For example, the body 210 may include a channel 214 that does not have an opening 216 and encircles the extension leg 162, guidewire 130, etc. completely. As such, the body 210 can slidably engage the portion of the catheter placement system 100 but cannot be detached therefrom. Advantageously, this prevents the catheter placement system 100 from accidently disengaging the device 200 and falling outside of the sterile field. Further, the device 200 can function to prevent a guidewire 130, for example, from being accidentally drawn through the catheter lumen and into the vasculature, mitigating complications to the patient.

In an embodiment, the channel 214 includes an opening 216, extending longitudinally and configured to allow ingress and/or egress of the portion of the extension leg 162 to/from the channel 214. In an embodiment, a width (w1) of the opening 216 is less than a diameter (d1) of the portion of the catheter placement system 100 (e.g., the extension leg 162) to be retained by the channel 214. As such, one or both of the opening 216 and the portion of the extension leg 162 elastically deforms slightly to allow the extension leg 162 to fit through opening 216. Further the opening 216 can abut against the extension leg 162 to retain the extension leg 162 within the channel 214. In an embodiment, a width of the channel 214 can be equal to, or smaller than, an outer diameter of the extension leg 162 and can engage the extension leg 162 in an interference fit, press-fit, or snap fit engagement to releasably retain the extension leg 162 therein.

In an exemplary method of use, a clinician can couple the device 200 to a portion of the catheter 150. For example, a first extension leg 162A is urged through a first opening 216A and into the first channel 214A, a second extension leg 162B is urged through a second opening 216B and into the first channel 214B, and a third extension leg 162C is urged through a third opening 216C and into the third channel 214C, or combinations thereof. Advantageously, the device 200 can retain the extension leg(s) 162, preventing them becoming tangled and preventing them from falling outside of the sterile field.

The clinician then places a digit 80, e.g., a little finger digit 80D, through the aperture 224 of the handle 220 to retain the device 200 and the extension legs 162, coupled thereto, proximate the clinician's hand and without compromising the dexterity of the clinician. Advantageously, this provides the clinician with increased control of the catheter placement system 100, e.g., the catheter 150 and guidewire 130. The clinician then accesses the vasculature with the needle 120 and the access section 154, confirm correct vascular access using the blood flash system 140, advance the guidewire 130, remove the needle 120, advance the catheter 150 over the guidewire 130, dilate the insertion site, place the distal tip 118 of the catheter 150 at a target location within the vasculature, and remove the guidewire 130, all without dropping the proximal end of the catheter placement system 100, risking contamination. Advantageously, the device 200 retains the proximal portions of the catheter placement system 100 away from the insertion site, providing improved visibility, a simplified presentation, and greater control.

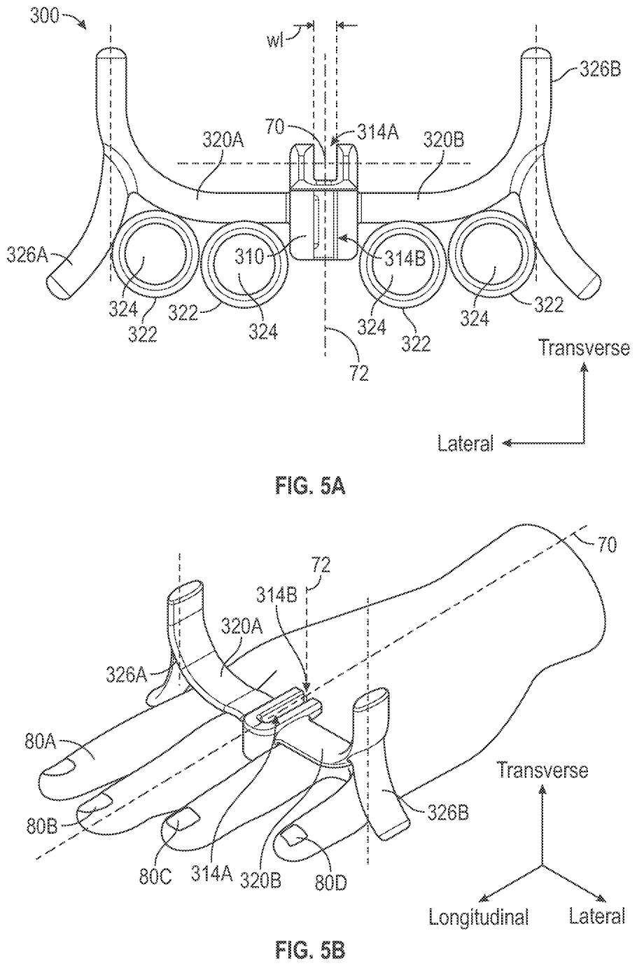

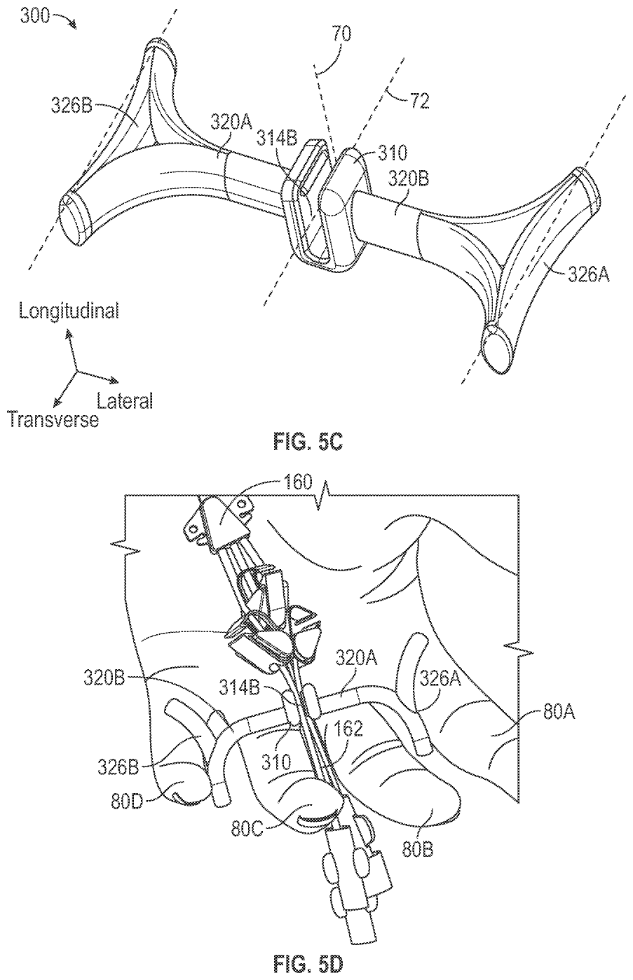

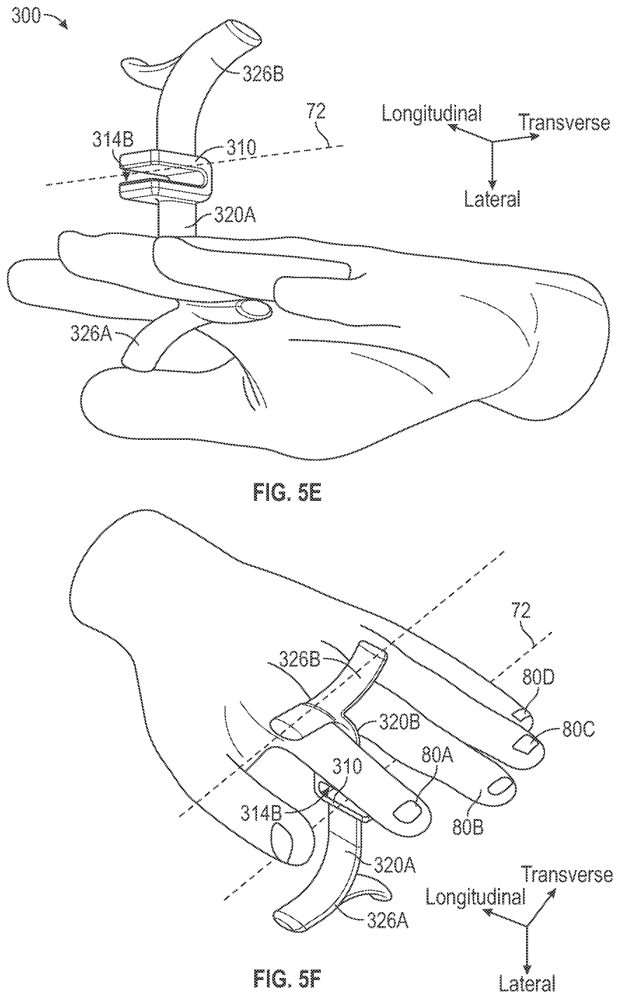

FIGS. 5A-5F show embodiments of a catheter containment device (“device”) 300. The device 300 generally includes a body 310 disposed centrally and includes a first handle 320A extending laterally from a first side of the body 310 and a second handle 320B extending laterally from a second side of the body 310, opposite the first side. Optionally the first handle 320A and/or the second handle 320B includes one or more rings 322 for placing a digit 80 therethrough, as described in more detail herein. For example, FIG. 5A shows an embodiment of the device 300 with one or more rings 322 and FIG. 5B shows an embodiment of the device 300 without any rings 322. As will be appreciated, these embodiments are exemplary and not intended to be limiting. The body 310 includes a first channel 314A extending along a central longitudinal axis 70 and configured to retain a portion of the catheter placement system 100, as described herein. As will be appreciated, the device 300 can include one or more first channels 314A each extending along a longitudinal axis 70 and each configured to retain a portion of the catheter placement system 100. However, for ease of illustration only a single longitudinal first channel 314A is shown. In an embodiment, the channel 314 can permanently retain a portion of the catheter placement system 100, as described herein, so as to prevent the device 300 from disengaging the catheter placement system 100. As described herein, the device 300 can slidably engage, or be permanently affixed to the catheter placement system 100.

As shown in FIGS. 5A-5C, in an embodiment, the body 310 further includes a second channel 314B, extending along an axis at an angle relative to the longitudinal axis 70. As shown, the second channel 314B can extend along a central transverse axis 72, perpendicular to the axis 70 of the first channel 314A. However, it will be appreciated that the second channel 314B can extend along an axis 72 extending at other angles relative to the first longitudinal axis 70. In an embodiment, the second channel 314B can be configured to retain a portion of the catheter placement system 100, as described herein. As will be appreciated, the device 300 can include one or more second channels 314B each extending along the (transverse) axis 72 and each configured to retain a portion of the catheter placement system 100, as described herein. However, for ease of illustration only a single transverse second channel 314B is shown. In an embodiment, one or both of the first channel 314A or the second channel 314B can include an opening defining a first width (w1) that is less than an outer diameter (d1) of the portion of the catheter placement system 100 retained therein, as described herein.

In an embodiment, one or both of the first handle 320A and the second handle 320B can include a cross bar 326, for example the first handle 320A includes a first cross bar 326A and the second handle 320B includes a second cross bar 326B. The crossbar 326 extends at an angle relative to an axis of the handle 320, for example, the cross bar 326 extends substantially perpendicular to the lateral axis of the handle 320. As shown, the cross bar 326 extends along a transverse axis, however, it will be appreciated that other angles and other axes are also contemplated. The cross bar 326 can define a linear or a curved shape and can be configured to be grasped by one or more digits 80 of the clinician. In an embodiment, as shown in FIG. 5C, the cross bar 326 can extend symmetrically from the handle 320. In an embodiment, as shown in FIG. 5A, the cross bar 326 can extend asymmetrically from the handle 320. Advantageously, the handle 320 and/or crossbar 326 are configured to be grasped by one or more digits 80 to facilitate manipulating the device 300 and a portion of the system 100 retained therein.

In an embodiment, the handle 320 is formed of a substantially rigid, or resilient material such as a plastic, polymer, metal, alloy, composite, or the like. In an embodiment, the handle 320 is formed of a flexible, malleable, or elastically deformable material such as a plastic, polymer, elastomer, rubber, silicone rubber, metal, alloy, shape memory material, super-elastic material, Nitinol, composite, or the like. In an embodiment, the handle 320 is formed of a first material and includes a second material disposed thereon. For example, the first material includes a substantially rigid material, as described herein and includes a second material disposed thereon, for example a softer material, elastomer, rubber, silicone rubber, or a material displaying a relatively high frictional co-efficient to facilitate grasping the handle 320.

As shown in FIG. 5A, in an embodiment, the handle 320 can further include a ring 322 coupled thereto, and defining an aperture 324 configured to receive one or more digits 80 therethrough, as described herein. In an embodiment, the handle 320 includes one or more rings 322, each configured to receive a digit 80 therethrough. As shown in FIG. 5A, the ring(s) 322 are coupled to the first handle 320A and the second handle 320B. However, the number, location and position of the one or more rings 322 shown in FIG. 5A is exemplary and not intended to be limiting. The ring(s) 322 can be formed of the same material as one or both of the handle 320 and the body 310. In an embodiment, the ring(s) 322 are formed of a different material from one or both of the handle 320 and the body 310 and coupled thereto using adhesive, bonding, welding, ultrasonic welding, or the like. Advantageously, the ring(s) 322 facilitate grasping the handle 320, securing the device 300 to the hand of the clinician, and mitigate accidental disengagement of the device 300 from the clinician's hand.

In an exemplary method of use, a clinician can grasp the device 300 by one or more of the body 310, first handle 320A, the second handle 320B, cross bar 326 and ring(s) 322, or combinations thereof. In an embodiment, the clinician can place one or more digits 80 through a ring 322, as described herein. In an embodiment, the clinician can grasp the device 300 by placing one or more digits 80 between the first cross bar 326A and the second cross bar 326B. In an embodiment, the clinician can grasp the device 300 with the handles 320 extending across a back of the fingers, i.e., a back side of the hand, (FIG. 5B). In an embodiment, a clinician can grasp the device 300 by applying pressure laterally outwards by the first digit 80A and the fourth digit 80D. In an embodiment, as shown in FIG. 5D, a portion of the first cross bar 326A can be grasped between a first digit 80A and a second digit 80A, and a portion of the second cross bar 326B can be grasped between a third digit 80C and a fourth digit 80D. In an embodiment, the clinician can grasp the device 300 with the handles 320 extending across a front of the fingers, i.e., a palm side of the hand. In an embodiment, a clinician can grasp the device 300 by applying pressure laterally inwards by the first digit 80A and the fourth digit 80D. Advantageously, the handles 320, crossbars 326, and/or rings 322 allow the clinician to grasp the device 300 in a variety of ways without impeding the dexterity of the hand used to grasp the device 300. The versatility of the device 300 allows the clinician to retain a proximal portion of the system 100 in a variety of positions as convenient to the clinician.

In an embodiment, as shown in FIGS. 5E-5F a clinician can grasp the device 300 by extending a handle 320 between two of the digits 80A-80D, for example between a first digit 80A and a second digit 80B, or a second digit 80B and a third digit 80C, or combinations thereof, etc. In an embodiment, the cross bar 326 extends across one of the front side of the fingers (i.e., palm side, FIG. 5E), or across a back side of the fingers (FIG. 5F). In an embodiment, the body 310 is disposed on one of the back side of the fingers (FIG. 5E), or the front side of the fingers (i.e., palm side, FIG. 5E). The clinician then uses one or both of the handle 320 and the cross bar 326 to lever the device 300 and facilitate manipulating the device 300 while maintaining the dexterity of the digits 80.

In an embodiment, the clinician can grasp the device 300, as described herein, and can place a portion of the catheter placement system 100 into either the first channel 314A to align the portion of the catheter placement system 100 with a longitudinal (first) axis 70, or the second channel 314B to align the portion of the catheter placement system 100 with a transverse (second) axis 72, depending on which position is most ergonomically convenient.

In an embodiment, one or both of the first channel 314A and the second channel 314B are configured to receive two or more portions of the catheter placement system 100 and retain these portions therein. For example, as shown in FIG. 5D, the second channel 314B retains a portion of the first extension leg 162A, the second extension leg 162B, and the third extension leg 162C. Advantageously, the device 300 allows the clinician to grasp the device 300 with a portion of the catheter placement system 100 secured therein and allows the clinician to maintain dexterity of the digits of the same hand. The device 300 allows the clinician to continue with the placement of the catheter 150 while mitigating the catheter 150 from falling outside of the sterile field. Further the device 300 prevents tangling of one or more proximal portions of the system 100.

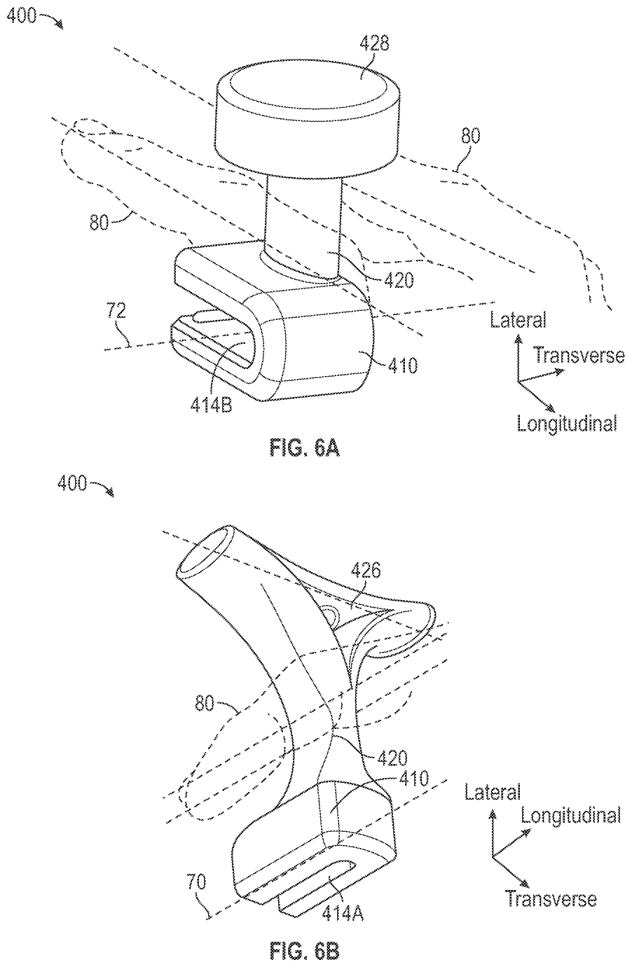

In an embodiment, as shown in FIGS. 6A-6B, a catheter containment device (“device”) 400 includes a body 410 and a single handle 420 extending therefrom. The handle 420 defines a circular or curvilinear cross-sectional shape. In an embodiment, the handle 420 includes a plug 428 (FIG. 6A), or a cross bar 426 (FIG. 6B) disposed at an opposite end of the handle 420 from the body 410. The plug 428 or the cross bar 426 can be configured to abut against the digits 80 and prevent the handle 420 from sliding therebetween. The body 410 includes one or both of a first channel 414A extending along a first (longitudinal) axis 70 (FIG. 6B), and a second channel 414B, extending along a second (transverse) axis 72 (FIG. 6A). It will be appreciated, however, that the body 410 can include one or more channels extending along other axes, or at angles thereto, and are contemplated to fall within the scope of the present invention. In an embodiment, the channel 414 is configured to releasably retain a portion of the catheter placement system 100, as described herein. In an embodiment, the channel 414 can permanently retain, slidably retain, and/or permanently affixed to a portion of the catheter placement system 100, as described herein.

In an exemplary method of use, a clinician can grasp the device 400 by one or more of the body 410, the handle 420, the cross bar 426, and the plug 428. In an embodiment, one or more of the handle 420 and the cross bar 426 extends between two or more digits 80. The body 410 can be aligned with either a back of the hand, or front (palm) side of the hand. In an embodiment, one of the plug 428 or the cross bar 426 can abut against a digit 80 and prevent the handle 420 from slipping between the digits 80. The clinician can then secure a portion of the catheter placement system 100 in one of the first channel 414A or the second channel 414B, as described herein.

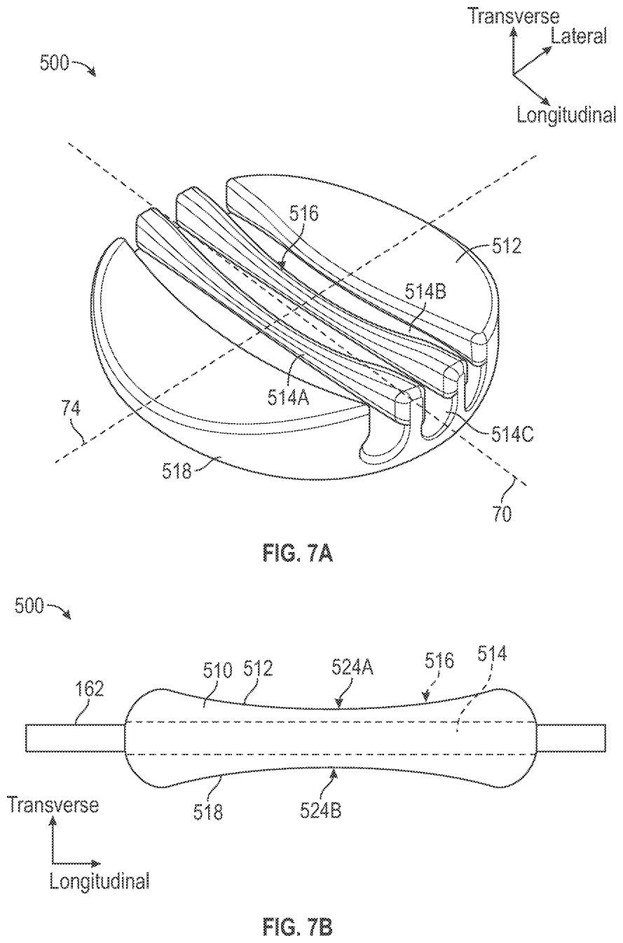

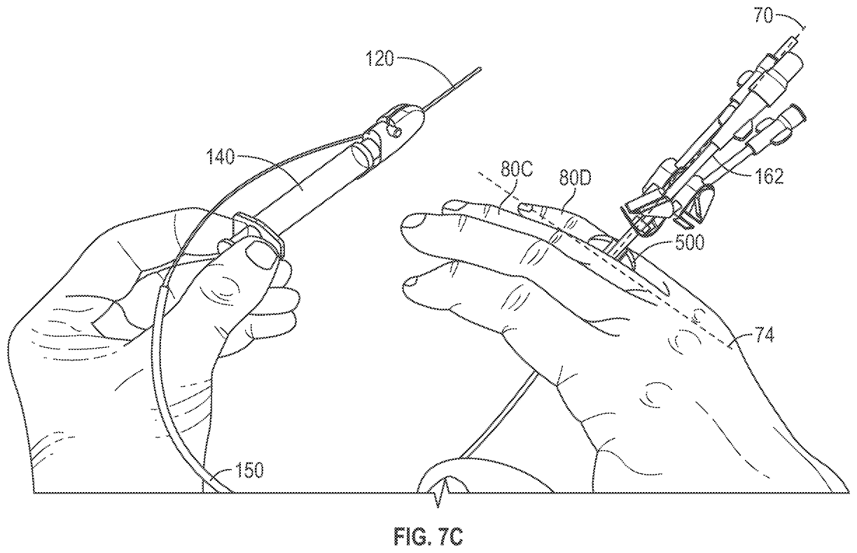

In an embodiment, as shown in FIGS. 7A-7C, a catheter containment device (“device”) 500 includes a body 510 substantially defining a disc shape extending over a horizontal plane and having a top surface 512, a bottom surface 518, and a circular outer perimeter. It will be appreciated, however, that the device 500 can define other three-dimensional outer profile shapes including cuboid, oval, polygonal, or any regular or irregular closed curve, convex or concave shapes. The body 510 of the device 500 includes one or more channels 514 extending longitudinally along a first axis 70, and configured to retain one or more portions of the catheter placement system 100, as described herein. The channel 514 can include an opening 516 communicating between the channel 514 and one of the top surface 512 or the bottom surface 518. In an embodiment, the channel 514 can permanently retain, slidably retain, and/or permanently affixed to a portion of the catheter placement system 100, as described herein.

FIG. 7B shows a lateral side view of the device 500. In an embodiment, one or both of the top surface 512 and the bottom surface 518 can define a concave profile. In an embodiment, the body 510 can define a bi-concave profile, or “hourglass” profile, having a concave top surface 512 and a concave bottom surface 518, disposed opposite the top surface 512. One or both of the concave top surface 512 and concave bottom surface 518 can each define a shallow finger channel 524 extending along a lateral axis 74. In use, the finger channel 524 can align a digit 80 of the clinician with an axis of the device 200 that extends at an angle, e.g., perpendicular, relative to the axis 70 of the channel 514.

In an embodiment, the device 500 can be formed of a substantially rigid, or resilient material such as a plastic, polymer, metal, alloy, composite, or the like. In an embodiment, the device 500 can be formed of a flexible, malleable, or elastically deformable material such as a plastic, polymer, elastomer, rubber, silicone rubber, metal, alloy, shape memory material, super-elastic material, Nitinol, composite, or the like. In an embodiment, the device 500 can be formed of a first material and can include a second material disposed thereon. For example, the first material includes a substantially rigid material, as described herein and includes a second material disposed thereon, for example a softer material, elastomer, rubber, silicone rubber, or a material displaying a relatively high frictional co-efficient to facilitate grasping the device 500 or securing a portion of the catheter placement system 100 therewith.

In use, as shown in FIG. 7C, the clinician can retain a portion of the catheter placement system 100 within a channel 514 of the device 500, in an interference fit, press-fit, snap-fit engagement, or the like, as described herein. The clinician can then grasp the device 500 between two digits 80, e.g., a third digit 80C and a fourth digit 80D, along a lateral axis 74, and retain the device 500 and catheter 150 assembly therebetween. Advantageously, the channel 514 can provide a large surface area between the device 500 and a portion of the catheter placement system 100, improving retention of the catheter placement system 100 in a friction fit engagement. Further, the device 500 can provide a relatively large surface area for a clinician to grasp the device 500, allowing the clinician to manipulate the device 500 and catheter placement system 100 coupled thereto, while mitigating slipping or dropping the portion of the catheter placement system 100.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

1. A catheter containment device for retaining a portion of a catheter placement system, comprising:
a body defining a channel extending longitudinally along a first axis, the channel configured to retain the portion of the catheter placement system, and having an opening extending longitudinally and configured to allow ingress or egress along a second axis extending at an angle to the first axis of the channel; and
a handle coupled to the body and configured to be grasped by a digit of a clinician.

2. The catheter containment device according to claim 1, wherein the portion of the catheter placement system includes one of a catheter body, catheter hub, extension leg, luer lock, or guidewire.

3. The catheter containment device according to claim 2, wherein the body includes a first channel configured to retain a first extension leg and a second channel configured to retain a second extension leg.

4. The catheter containment device according to claim 2, wherein the body includes a first channel configured to retain a first extension leg and a second extension leg.

5. The catheter containment device according to claim 1, wherein the body includes a first channel extending along the first axis of the channel, and a second channel extending along a third axis at an angle to the first axis of the channel.

6. The catheter containment device according to claim 1, wherein an inner diameter of the channel is equal to or slightly smaller than an outer diameter of the portion of the catheter placement system.

7. The catheter containment device according to claim 1, wherein a width of the opening of the channel is smaller than an outer diameter of the portion of the catheter placement system.

8. The catheter containment device according to claim 1, wherein one or both of the body and the handle is formed of a substantially rigid, or resilient material selected from a group consisting of a plastic, polymer, metal, alloy, or composite.

9. The catheter containment device according to claim 1, wherein one or both of the body and the handle is formed of a flexible, malleable, or elastically deformable material selected from a group consisting of a plastic, polymer, elastomer, rubber, silicone rubber, metal, alloy, shape memory material, super-elastic material, Nitinol, composite.

10. The catheter containment device according to claim 1, wherein the handle is formed of a first material having a substantially rigid, or resilient, material properties and including a second material disposed thereon having a relatively softer, or more flexible material properties.

11. The catheter containment device according to claim 1, wherein the handle is a ring defining an aperture and configured to receive one or more digits therethrough.

12. The catheter containment device according to claim 1, wherein the handle is hingedly, rotatably or pivotally coupled to the body.

13. The catheter containment device according to claim 1, wherein the handle extends from the body at an angle relative to the first axis of the channel.

14. The catheter containment device according to claim 1, further including a first handle extending from the body in a first direction and a second handle extending from the body in a second direction, opposite the first direction.

15. The catheter containment device according to claim 1, wherein the handle includes one of a plug or a cross bar disposed at an opposite end of the handle from the body.

16. A catheter containment device for retaining a portion of a medical device, comprising:
a body defining a disc shape extending over a horizontal plane and defining a top surface and a bottom surface; and
a device channel extending along an axis, parallel to one of the top surface or the bottom surface, and including an opening communicating with one of the top surface or the bottom surface, the device channel configured to receive a portion of the medical device therein.

17. The catheter containment device according to claim 16, wherein the body further includes a bi-concave cross-section having one of a first finger channel extending over the top surface and a second finger channel extending over the bottom surface, one of the first finger channel or the second finger channel extending at an angle relative to the device channel.

18. The catheter containment device according to claim 16, wherein the device channel is configured to retain one of a catheter body, catheter hub, extension leg, luer lock, or guidewire.

19. The catheter containment device according to claim 16, wherein the body includes a first device channel configured to retain a first extension leg and a second device channel extending parallel to the first device channel and configured to retain a second extension leg.

20. The catheter containment device according to claim 16, wherein the first device channel is configured to retain a first extension leg and a second extension leg.

21. The catheter containment device according to claim 16, wherein an inner diameter of the device channel is equal to or slightly smaller than an outer diameter of the portion of the medical device.

22. The catheter containment device according to claim 16, wherein a width of the opening of the device channel is smaller than an outer diameter of the portion of the medical device.

23. The catheter containment device according to claim 16, wherein the body is formed of a substantially rigid, or resilient material selected from a group consisting of a plastic, polymer, metal, alloy, or composite.

24. The catheter containment device according to claim 16, wherein the body is formed of a flexible, malleable, or elastically deformable material selected from a group consisting of a plastic, polymer, elastomer, rubber, silicone rubber, metal, alloy, shape memory material, super-elastic material, Nitinol, composite.

25. The catheter containment device according to claim 16, wherein the body is formed of a first material having a substantially rigid, or resilient, material properties and including a second material disposed thereon having a relatively softer, or more flexible material properties.

26-37. (canceled)
